(19) United States
(12) Patent Application Publication
(10) Pub. No.: US 2026/0102126 A1
(43) Pub. Date: Apr. 16, 2026

(54) AUTONOMOUS DETECTION AND ADJUSTMENT RELATING TO DEVICE FLIPPING AND SENSOR NOISE

(71) Applicant: Medtronic, Inc., Minneapolis, MN (US)

(72) Inventors: Nicholas H. Finstrom, Coon Rapids, MN (US); Mark R. Boone, Gilbert, AZ (US); Aaron B. Bartnik, Osseo, MN (US); Patrick M. Hermans, Lake Elmo, MN (US)

(21) Appl. No.: 19/341,101

(22) Filed: Sep. 26, 2025

Related U.S. Application Data
(60) Provisional application No. 63/705,961, filed on Oct. 10, 2024.

(51) Int. Cl. A61B 5/00; A61B 5/026; A61B 5/06; A61B 34/20; G16H 40/67

(52) U.S. Cl. CPC A61B 5/7285; A61B 5/0261; A61B 5/067; A61B 5/068; A61B 5/6861; A61B 34/20; G16H 40/67; A61B 2034/2055

(57) ABSTRACT

Various embodiments relate to system, methods, and storage media for autonomous detection and adjustment relating to device flipping and sensor noise for an implanted optical sensing device (e.g., implanted cardiac monitoring device configured to provide a photoplethysmography signal). In some variations, a method of operating an optical device implanted in a patient includes receiving at least one sensor signal from one or more sensors of an implanted optical device, detecting an orientation and/or optical noise condition of the optical device based on the at least one sensor signal, and modifying one or more characteristics of optical circuitry associated with an optical sensor arrangement in the optical device, based on the detected orientation of the device and/or the detected optical noise condition.

### RELATED APPLICATIONS

This application claims the benefit of U.S. Provisional Application Ser. No. 63/705,961, filed Oct. 10, 2024, the entire contents of which is incorporated herein by reference.

### TECHNICAL FIELD

The present technology relates to the autonomous detection and/or adjustment relating to device flipping and sensor noise for an implanted device.

### BACKGROUND

Many medical devices (e.g., pulse oximeters) utilize photoplethysmography (PPG), an optical technique that can be used to detect volumetric changes in blood in peripheral circulation. For example, PPG can be used to detect blood volume changes in the microvascular bed of tissue. PPG can, for example, provide quantification of physiological metrics such as blood pressure, heart rate, and blood oxygenation levels.

In general, PPG involves operation of an optical emitter-detector pair, where the emitter is a light source that illuminates tissue of interest, and the detector operates in a reflectance or transmittance mode to measure the amount of that light that is reflected or otherwise transmitted to the detector. Some PPG devices are external (e.g., wearable), while some PPG devices are implantable in a patient.

However, in instances in which the PPG device is implanted in a patient, the signal strength of the device's PPG signal may be subject to variation due to patient-to-patient differences, as well as changes in position and/or orientation of the optical sensing device once placed at a target implant site. Furthermore, optical signals obtained with the detector may be subject to noise from external, ambient light sources that can interfere with the optical signals of interest.

### SUMMARY

The subject technology is illustrated, for example, according to various aspects described below, including with reference to FIG. 1-7. Various examples of aspects of the subject technology are described as numbered clauses (1, 2, 3, etc.) for convenience. These are provided as examples and do not limit the subject technology.

Various embodiments described herein relate to a method of operating an optical device implanted in a patient, the method including one more of the following: receiving at least one sensor signal from one or more sensors of the implanted optical device; detecting an orientation of the optical device based on the least one sensor signal; and based on the detected orientation of the optical device, modifying one or more characteristics of optical circuitry associated with an optical sensor arrangement in the optical device, wherein the optical sensor arrangement is configured to generate a photoplethysmography (PPG) signal for the patient.

Various embodiments are described wherein the at least one sensor signal comprises an optical sensor signal from the optical sensor arrangement of the optical device.

Various embodiments are described wherein the optical sensor signal comprises a photoplethysmography (PPG) signal, and wherein detecting an orientation of the optical device comprises detecting an orientation of the optical device based on the PPG signal.

Various embodiments are described wherein detecting an orientation of the optical device comprises evaluating ambient light level outside of the patient based on the at least one optical sensor signal.

Various embodiments are described wherein the one or more sensors comprises an impedance sensor, wherein the at least one sensor signal comprises an impedance sensor signal from the impedance sensor, and wherein detecting an orientation of the optical device comprises evaluating a type of tissue contacting the optical device based on the impedance sensor signal.

Various embodiments are described wherein receiving at least one sensor signal comprises receiving an accelerometer signal, and wherein the method further comprises evaluating optical sensing conditions based on the accelerometer signal.

Various embodiments are described wherein modifying one or more characteristics of optical circuitry comprises modifying a drive current associated with an emitter of the optical sensor arrangement.

Various embodiments are described wherein modifying one or more characteristics of optical circuitry comprises modifying an integration capacitor value in an integrator amplifier of the optical circuitry.

Various embodiments are described wherein modifying one or more characteristics of optical circuitry comprises modifying an integration time value in an integrator amplifier of the optical circuitry.

Various embodiments described herein additionally include generating a measurement PPG signal for the patient using the modified optical circuitry.

Various embodiments are described wherein the optical device comprises an implantable cardiac monitoring device.

Various embodiments described herein relate to an implantable cardiac monitoring device, including one or more of the following: one or more sensors; a processor; a memory operably coupled to the processor and storing instructions that, when executed by the processor, cause the cardiac monitoring device to perform operations comprising: receiving at least one sensor signal from the one or more sensors when the cardiac monitoring device is implanted in a patient; detecting an orientation of the cardiac monitoring device based on the least one sensor signal; and based on the detected orientation of the cardiac monitoring device, modifying one or more characteristics of optical circuitry associated with an optical sensor arrangement in the cardiac monitoring device, wherein the optical sensor arrangement is configured to generate a photoplethysmography (PPG) signal for the patient.

Various embodiments are described wherein the at least one sensor signal comprises an optical sensor signal from the optical sensor arrangement of the cardiac monitoring device.

Various embodiments are described wherein the optical sensor signal comprises a photoplethysmography (PPG) signal, and wherein detecting an orientation of the cardiac monitoring device comprises detecting an orientation of the cardiac monitoring device based on the PPG signal.

Various embodiments are described wherein detecting an orientation of the cardiac monitoring device comprises evaluating ambient light level outside of the patient based on the at least one optical sensor signal.

Various embodiments are described wherein the one or more sensors comprises an impedance sensor, wherein the at least one sensor signal comprises an impedance sensor signal from the impedance sensor, and wherein detecting an orientation of the cardiac monitoring device comprises evaluating a type of tissue contacting the cardiac monitoring device based on the impedance sensor signal.

Various embodiments are described wherein receiving at least one sensor signal comprises receiving an accelerometer signal, and wherein the method further comprises evaluating optical sensing conditions based on the accelerometer signal.

Various embodiments are described wherein modifying one or more characteristics of optical circuitry comprises modifying a drive current associated with an emitter of the optical sensor arrangement.

Various embodiments are described wherein modifying one or more characteristics of optical circuitry comprises modifying an integration capacitor value in an integrator amplifier of the optical circuitry.

Various embodiments are described wherein modifying one or more characteristics of optical circuitry comprises modifying an integration time value in an integrator amplifier of the optical circuitry.

Various embodiments described herein additionally include generating a measurement PPG signal for the patient using the modified optical circuitry.

Various embodiments described herein relate to a method of operating an optical device implanted in a patient, the method including one or more of the following: receiving at least one optical sensor signal from an optical sensor arrangement of the implanted optical device, wherein the optical sensor arrangement is configured to generate a photoplethysmography (PPG) signal for the patient; detecting an optical noise condition for the optical device based on the least one optical sensor signal; and based on the detected optical noise condition of the optical device, modifying one or more characteristics of optical circuitry associated with the optical sensor arrangement.

Various embodiments are described wherein the optical noise condition is due to ambient light, signal attenuation, or both.

Various embodiments are described wherein modifying one or more characteristics of optical circuitry comprises modifying a drive current associated with an emitter of the optical sensor arrangement.

Various embodiments are described wherein modifying one or more characteristics of optical circuitry comprises modifying an integration capacitor value in an integrator amplifier of the optical circuitry.

Various embodiments are described wherein modifying one or more characteristics of optical circuitry comprises modifying an integration time value in an integrator amplifier of the optical circuitry.

Various embodiments described herein additionally include generating a measurement PPG signal for the patient using the modified optical circuitry.

Various embodiments are described wherein the optical device comprises a cardiac monitoring device.

Various embodiments described herein relate to an implantable cardiac monitoring device, including one or more of the following: an optical sensor arrangement, wherein the optical sensor arrangement is configured to generate a photoplethysmography (PPG) signal for the patient; a processor; a memory operably coupled to the processor and storing instructions that, when executed by the processor, cause the cardiac monitoring device to perform operations comprising: receiving at least one optical sensor signal from the optical sensor arrangement; detecting an optical noise condition for the cardiac monitoring device based on the least one optical sensor signal; and based on the detected optical noise condition of the cardiac monitoring device, modifying one or more characteristics of optical circuitry associated with the optical sensor arrangement.

Various embodiments are described wherein the optical noise condition is due to ambient light, signal attenuation, or both.

Various embodiments are described wherein modifying one or more characteristics of optical circuitry comprises modifying a drive current associated with an emitter of the optical sensor arrangement.

Various embodiments are described wherein modifying one or more characteristics of optical circuitry comprises modifying an integration capacitor value in an integrator amplifier of the optical circuitry.

Various embodiments are described wherein modifying one or more characteristics of optical circuitry comprises modifying an integration time value in an integrator amplifier of the optical circuitry.

Various embodiments described herein additionally include generating a measurement PPG signal for the patient using the modified optical circuitry.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a schematic flowchart of an example variation of a method for operating an optical sensing device having multiple optical vectors for generating a PPG signal, in accordance with the present technology.

FIG. 2A is an illustrative schematic of an example optical device, in accordance with the present technology.

FIG. 2B is an illustrative schematic of example circuitry for processing signals from an optical sensor, in accordance with the present technology.

FIG. 3 is an illustrative schematic of an example variation of an insertable cardiac monitoring device, in accordance with the present technology.

FIG. 4 is a conceptual diagram of an example variation of an insertable cardiac monitoring device, in accordance with the present technology.

FIG. 5 is an illustrative schematic of a cardiac monitoring system including an insertable cardiac monitoring device placed in a patient, in accordance with the present technology.

FIGS. 6A and 6B are illustrative schematics of use of impedance sensing to determine device orientation, in accordance with the present technology.

FIG. 7 is a schematic flowchart of an example variation of a method for dynamically self-tuning optical circuitry settings for an optical sensor arrangement, in accordance with the present technology.

### DETAILED DESCRIPTION

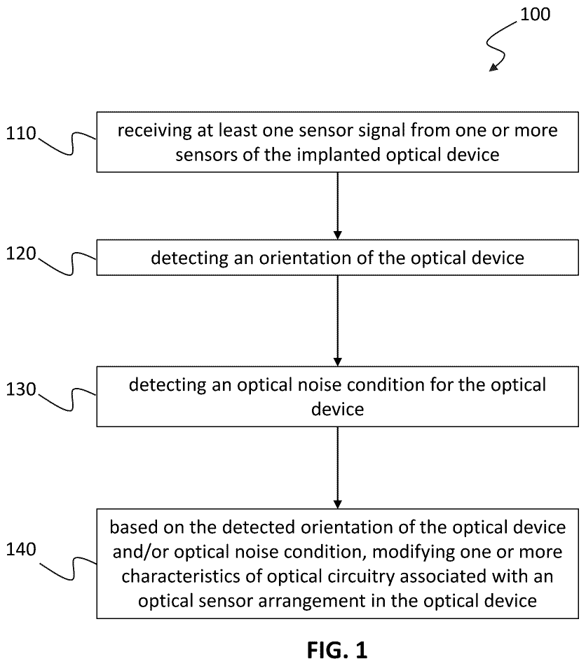

The present technology relates to systems and methods enabling implanted optical devices to self-detect device orientation (e.g., device flipping) and/or optical noise conditions, and/or to self-adjust sensing configurations to compensate for device orientation and/or optical noise conditions. Some variations of the present technology, for example, are directed to implantable cardiac monitoring devices and methods of operating such devices. Specific details of several variations of the technology are described below with reference to FIG. 1-7.

Optical sensing devices for generating PPG signals may be subject to variation and/or noise due to the environment in which the optical sensing device is placed. For example, PPG or other optical signals obtained from a reflectance mode optical sensor in an implanted medical device may be subject to variation in signal strength due at least in part to local anatomy at the implant site, which may vary from patient to patient, as well as changes in position and/or orientation of the optical sensing device in the patient. Furthermore, external or ambient light sources may interfere with the optical signals of interest, thereby creating noise in the desired PPG signal.

Described herein are techniques for optical sensing devices (also referred to herein as “optical devices”) to self-detect device orientation while implanted in a patient, and/or self-adjusting various one or more characteristics of optical sensing to improve a measurement PPG signal in view of device orientation and/or optical noise (e.g., due to ambient light level, tissue surrounding the implanted optical sensing devices, etc.). Generally, such techniques involve receiving sensor information from one or more sensors in the implanted optical sensing device such as one or more optical sensors and/or one or more sensors concurrent with the optical sensors (e.g., accelerometer, impedance sensor, etc.), and/or one or more sensors in an external wearable device (e.g., accelerometer). The sensor data may be analyzed to determine an orientation of the implanted optical device and/or an optical noise condition (and/or other device status). Various characteristics of optical circuitry associated with the optical sensor arrangement may be adjusted in response to determination of the device orientation and/or optical noise condition.

For example, as shown in FIG. 1, a method 100 may include receiving at least one sensor signal from one or more sensors of an implanted optical device 110, detecting an orientation of the optical device based on the at least one sensor signal 120, and detecting an optical noise condition for the optical device 130. Although FIG. 1 illustrates both detecting an orientation of the optical device 120 and detecting an optical noise condition for the optical device 130, it should be understood that in some variations, the method 100 may include detecting an orientation of the optical device 120 without detecting an optical noise condition for the optical device 130, or may include detecting an optical noise condition for the optical device 130 without detecting an orientation of the optical device 120. The method 10 may further include modifying one or more characteristics of optical circuitry associated with an optical sensor arrangement in the optical device 140, based on the detected orientation of the device and/or the detected optical noise condition. Further aspects of the method 100 are described in detail herein.

### I. Optical Devices and Systems

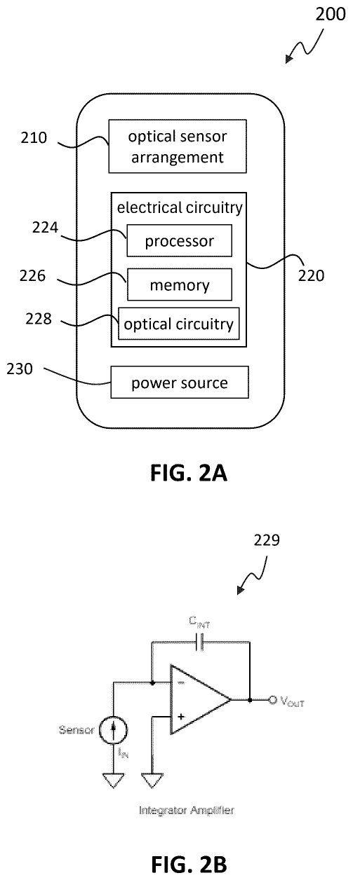

The techniques described herein may be used in various optical sensing devices configured to generate a PPG signal using one or more optical detectors and, optionally, one or more optical emitters. For example, FIG. 2A is an illustrative schematic of an example optical sensing device 200, which includes an optical sensor arrangement 210, electrical circuitry 220, and a power source 230.

The optical sensor arrangement 210 may include an emitter set of one or more emitters and a detector set of one or more detectors. The emitter set and the detector set may, in some variations, be located under an optically transparent surface (e.g., sapphire, glass, ceramic, device header) of the optical sensing device, so as to allow passage of light out of and into the optical sensing device. Each emitter may be configured, when activated, to emit light at a desired wavelength suitable for PPG measurements. For example, an emitter in the optical sensor arrangement 210 may be configured to emit light at a red wavelength (e.g., 640 nm-660 nm, or about 660 nm), a green wavelength (e.g., 530 nm-550 nm, or about 550 nm), or an infrared wavelength (e.g., 880 nm-940 nm, or about 940 nm). In some variations, light emitted from an active emitter may be filtered with one or more suitable filters in the optical pathway between the emitter and a corresponding detector in an emitter-detector pair being used to generate a PPG signal. Such filter(s) may block wavelengths that are not of interest for illuminating tissue, and pass through wavelengths that are of interest for illuminating tissue. For example, the filter(s) may include one or more suitable low-pass filters, high-pass filters, bandpass filters, or bandstop filters. The filter(s) may be coupled to an output side of the emitter or otherwise located in the optical pathway between the emitter and the detector. The emitter set may include any suitable type of light source, including, for example, a light-emitting diode (LED). Furthermore, each detector in the detector set may be configured to detect light that is emitted from an emitter and reflected off tissue of interest, where a PPG signal may be derived from measurements of the reflected light. For example, a detector in the optical sensor arrangement 210 may include a photodiode. In some variations, the optical sensor arrangement 210 may include multiple optical pathways defined by different emitter-detector pair locations. For example, a first emitter-detector pair may be on a first surface of the optical sensing device configured to be facing outward from the patient, while a second emitter-detector may be on a second surface of the optical sensing device configured to be facing inwards into the patient, where the first and second surfaces are opposite each other. However, different emitter-detector pairs may be located on various suitable surfaces of the optical sensing device. In some variations, the optical sensor arrangement 210 may include zero emitters within the implanted device and instead rely on light sources external to the optical sensing device 200, such as ambient lighting or a second implantable or external device containing one or more emitters. In these variations, the optical pathway driving PPG signals will generally be transmissive instead of reflective; however, the physiological pressure dependent dynamic optical attenuation used to generate PPG signals are the same.

Electrical circuitry 220 may include any discrete and/or integrated electronic circuit components that implement analog and/or digital circuits capable of producing the functions described for operating the optical sensor arrangement 210 (e.g., activating the emitter(s) in the emitter set) and/or generating a PPG signal. For example, the electrical circuitry 220 may include analog circuits, e.g., pre-amplification circuits, filtering circuits, and/or other analog signal conditioning circuits. For example, the electrical circuitry 220 may include optical circuitry 228 configured for processing one or more sensor outputs from the detector set. For example, FIG. 2B illustrates a representative example of an integrator amplifier circuit 229 that is configured to translate the current (I_{N}) from an optical detector into a voltage waveform (V_{OUT}) that is measurable by an analog-to-digital converter. More specifically, the circuit 229 includes an integrating capacitor configured to integrate the detector current I_{N} over a suitable integration time. The capacitor may have a fixed or variable capacitance value, and the integration time may be fixed or adjustable. Thus, in some variations, the capacitance value and/or the integration time may be adjusted (e.g., via other components of electrical circuitry 220) to vary the gain and/or attenuation factor of the detector signal. Additionally or alternatively, the signal current for the detector may be sampled for more time (e.g., as controlled by other components of electrical circuitry 220), which may result in an increase of the gain and signal-to-noise ratio (SNR) of the PPG signal, with the tradeoff of lower available sampling rate.

The electrical circuitry 220 and/or other portions of the device 200 may also include digital circuits, e.g., digital filters, combinational or sequential logic circuits, state machines, integrated circuits, one or more processors 224 (shared, dedicated, or group) that executes one or more software or firmware programs, memory devices 226, or any other suitable components or combination thereof that provide the described functionality.

Power source 230 provides power to electrical circuitry 220, the optical sensor arrangement 210, as well as to any other components that require power. Power source 230 may include one or more energy storage devices, such as one or more rechargeable or non-rechargeable batteries.

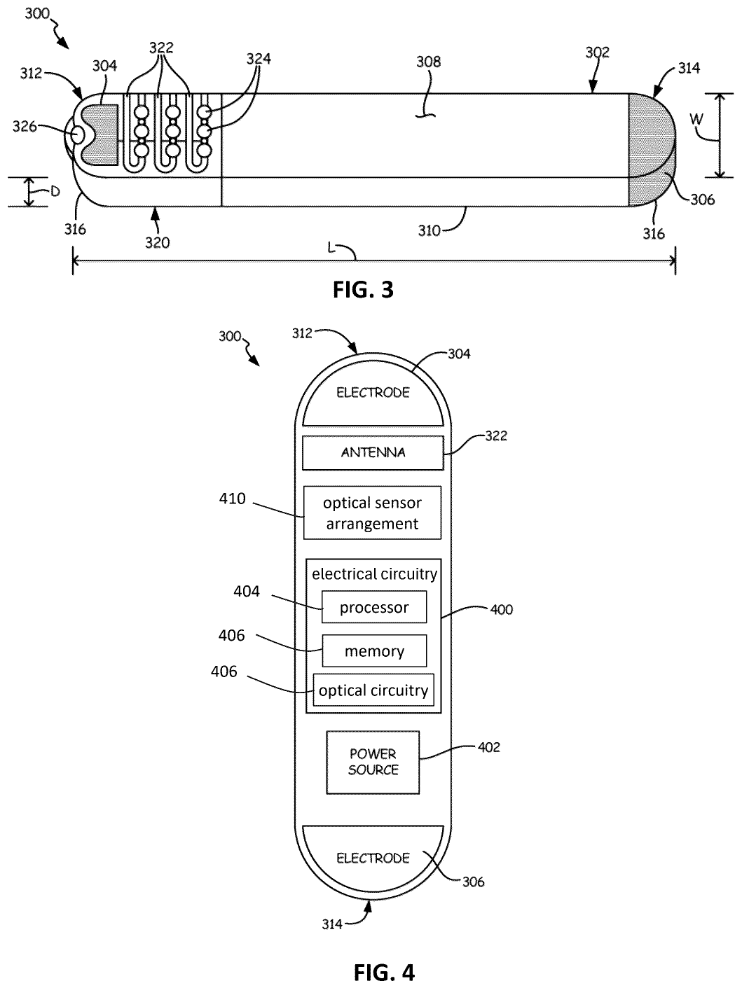

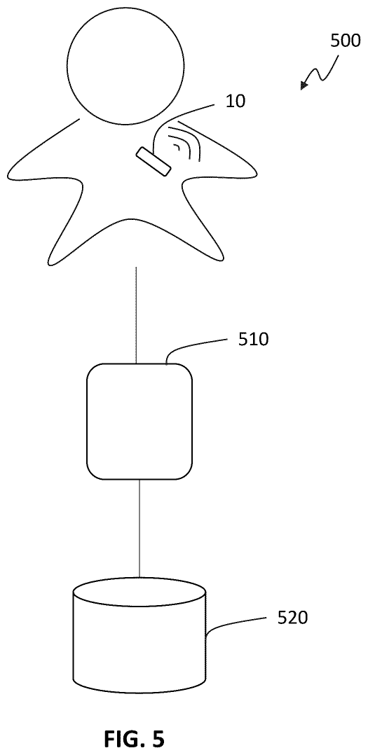

In some variations, the techniques described herein for using multiple optical vectors for PPG signal optimization can be performed with respect to generating a PPG signal with an implantable device such as an insertable cardiac monitor. FIG. 3 is a conceptual diagram of an example of an insertable cardiac monitor (ICM) 300 (also referred to herein as a “cardiac monitoring device”) for detecting a bradycardia/asystole event, according to another variation of the present disclosure. Insertable cardiac monitor 300 is an example of optical sensing device 200. In the example shown in FIG. 3, insertable cardiac monitor 300 may be embodied as a monitoring device having housing 302, a first (e.g., proximal) electrode 304, and a second (e.g., distal) electrode 306. Housing 302 may further comprise a first major surface 308, a second major surface 310, a first (e.g., proximal) end 312, and a second (e.g., distal) end 314. Housing 302 encloses electronic circuitry 400 and power source 402 (shown in FIG. 4) located inside the insertable cardiac monitor 300 and protects the circuitry contained therein from body fluids. Electrical feedthroughs provide electrical connection of electrodes 304 and 306.

In some variations such as that shown in FIG. 3, insertable cardiac monitor 300 is defined by a length L, a width W and thickness or depth D. The ICM may be in the form of an elongated rectangular prism wherein the length L is much larger than the width W, which in turn is larger than the depth D. In some variations, the geometry of the insertable cardiac monitor 300 (for example, a width W greater than the depth D) may be selected to allow the cardiac monitor 300 to be inserted under the skin of the patient using a minimally invasive procedure and to remain in the desired orientation during insert. For example, the device shown in FIG. 3 may include radial asymmetries (notably, the rectangular shape) along the longitudinal axis that maintains the device in the proper orientation following insertion. For example, in some variations the spacing between the proximal electrode 304 and distal electrode 306 may range from 30 millimeters (mm) to 55 mm, 35 mm to 55 mm, and from 40 mm to 55 mm and may be any range or individual spacing from 25 mm to 60 mm. In addition, insertable cardiac monitor 300 may have a length L that ranges from 30 mm to about 70 mm. In other variations, the length L may range from 40 mm to 60 mm, 45 mm to 60 mm and may be any length or range of lengths between about 30 mm and about 70 mm. In addition, the width W of major surface 308 may range from 3 mm to 10 mm and may be any single or range of widths between 3 mm and 10 mm. In some variations, the thickness of depth D of the insertable cardiac monitor 300 may range from 2 mm to 9 mm. For example, the depth D of the insertable cardiac monitor 300 may range from 2 mm to 5 mm and may be any single or range of depths from 2 mm to 9 mm. In addition, insertable cardiac monitor 300 according to an example variation of the present invention has a geometry and size designed for ease of implant and patient comfort. Variations of insertable cardiac monitor 300 described in this disclosure may have a volume of three cubic centimeters (cm) or less, 1.5 cubic cm or less or any volume between three and 1.5 cubic centimeters.

In the example shown in FIG. 3, once inserted within the patient, the first major surface 308 faces outward, toward the skin of the patient while the second major surface 310 is located opposite the first major surface 308. In addition, in the example shown in FIG. 3, proximal end 312 and distal end 314 are rounded to reduce discomfort and irritation to surrounding tissue once inserted under the skin of the patient. Insertable cardiac monitor 300, including instrument and method for inserting monitor 300 is described, for example, in U.S. Patent Publication No. 2014/0276928, incorporated herein by reference in its entirety.

As described with other variations, proximal electrode 304 and distal electrode 306 may be used to sense cardiac signals for determining a cardiac event (e.g., bradycardia or asystole event) such as EGM signals, intra-thoracically or extra-thoracically, which may be sub-muscularly or subcutaneously. EGM signals may be stored in a memory of the insertable cardiac monitor 300, and EGM data may be transmitted via integrated antenna 322 to another medical device, which may be another implantable device or an external device.

In the example variation shown in FIG. 3, proximal electrode 304 is in close proximity to the proximal end 312 and distal electrode 306 is in close proximity to distal end 314. In this variation, distal electrode 306 is not limited to a flattened, outward facing surface, but may extend from first major surface 308 around rounded edges 316 and onto the second major surface 310 so that the electrode 306 has a three-dimensional curved configuration. In the example variation shown in FIG. 3, proximal electrode 304 is located on first major surface 308 and is substantially flat, outward facing. However, in other variations, proximal electrode 304 may utilize the three-dimensional curved configuration similar to that of distal electrode 306, providing a three-dimensional proximal electrode (not shown in this variation). Additionally or alternatively, in other variations, distal electrode 306 may utilize a substantially flat, outward facing electrode located on first major surface 308 similar to that shown with respect to proximal electrode 304. The various electrode configurations allow for configurations in which proximal electrode 304 and distal electrode 306 are located on both first major surface 308 and second major surface 310. In other configurations, such as that shown in FIG. 3, only one of proximal electrode 304 and distal electrode 306 is located on both major surfaces 308 and 310, and in still other configurations both proximal electrode 304 and distal electrode 306 are located on one of the first major surface 308 or the second major surface 310 (e.g., proximal electrode 304 located on first major surface 308 while distal electrode 306 is located on second major surface 310). In some variations, the insertable cardiac monitor 300 may include electrodes on both major surface 308 and 310 at or near the proximal and distal ends of the device, such that a total of at least four electrodes are included on cardiac monitor device 300. Electrodes 304 and 306 may be formed of a plurality of different types of biocompatible conductive material (e.g. stainless steel, titanium, platinum, iridium, or alloys thereof), and/or may utilize one or more coatings such as titanium nitride or fractal titanium nitride.

In the example shown in FIG. 3, proximal end 312 includes a header assembly 320 that includes one or more of proximal electrode 304, an integrated antenna 322, anti-migration projections 324, and/or suture hole 326. The integrated antenna 322 may be located on the same major surface (e.g., first major surface 308) as proximal electrode 304 and may also be included as part of header assembly 320. Integrated antenna 322 allows insertable cardiac monitor 300 to transmit and/or receive data. In some variations, integrated antenna 322 may be formed on the opposite major surface as proximal electrode 304, or may be incorporated within the housing 322 of insertable cardiac monitor 300. In the example variation shown in FIG. 3, anti-migration projections 324 are located adjacent to integrated antenna 322 and protrude away from first major surface 308 to prevent longitudinal movement of the device, though may be arranged on any suitable surface of the insertable cardiac monitor 300. In the example variation shown in FIG. 3, anti-migration projections 324 include a plurality (e.g., nine) small bumps or protrusions extending away from first major surface 308; however, anti-migration projections 324 may additionally or alternatively be located on the opposite major surface as proximal electrode 304 and/or integrated antenna 322. As shown in FIG. 3, the suture hole 326, which may be used to help secure the insertable cardiac monitor 300 in the patient to prevent movement following insertion of the insertable cardiac monitor 300, may be located adjacent to proximal electrode 304, though one or more suture holes 326 may additionally or alternatively be located in any other suitable location. In some variations, the header assembly 320 is a molded header assembly made from a polymeric or plastic material, which may be integrated or separable from the main portion of insertable cardiac monitor 300.

FIG. 4 is a functional schematic diagram of the insertable cardiac monitor 300 as shown in FIG. 3 in accordance with the present technology. Insertable cardiac monitor 300 includes housing 302, proximal electrode 304 located at proximal end 312, distal electrode 306 located at distal end 314, integrated antenna 322, electrical circuitry 400 (which is an example of electrical circuitry 220), and power source 402 (which is an example of power source 230). In some variations, the insertable cardiac monitor 300 may include an optical sensor arrangement 410 (an example of optical sensor arrangement 210) comprising an emitter set of one or more optical light emitters and a detector set of one or more optical light detectors. The optical sensor arrangement 410 may, for example, be configured to provide a photoplethysmography (PPG) signal using the emitter and detector sets.

Electrical circuitry 400 may be coupled to proximal electrode 304 and distal electrode 306 to sense cardiac signals and monitor events (e.g., arrythmia, etc.). Electrical circuitry 400 is also connected to transmit and receive communications via integrated antenna 322. Power source 402 provides power to electrical circuitry 400, as well as to any other components that require power. Power source 402 may include one or more energy storage devices, such as one or more rechargeable or non-rechargeable batteries. The insertable cardiac monitor 300 as shown in FIGS. 3 and 4 may be a monitoring-only device. However, in other examples, insertable cardiac monitor 300 may further provide therapy delivery capabilities.

The electrical circuitry 400 receives raw EGM signals monitored by proximal electrode 304 and distal electrode 306. Electrical circuitry 400 may also include components/modules for converting the raw EGM signal to a processed EGM signal that can be analyzed to detect sense events. Although not shown, electrical circuitry 400 may include any discrete and/or integrated electronic circuit components that implement analog and/or digital circuits capable of producing the functions described for analyzing EGM signals to detect/verify bradycardia and/or asystole events. For example, the electrical circuitry 400 may include analog circuits, e.g., pre-amplification circuits, filtering circuits, and/or other analog signal conditioning circuits. The modules may also include digital circuits, e.g., digital filters, combinational or sequential logic circuits, state machines, integrated circuits, one or more processors 404 (shared, dedicated, or group) that executes one or more software or firmware programs, memory devices 406, or any other suitable components or combination thereof that provide the described functionality.

In some variations, electrical circuitry 400 may include a sensing unit for monitoring the EGM signal detected by the respective proximal and distal electrodes 304 and 306, respectively, and at least one sensing channel that utilizes an algorithm for identifying events in the EGM signal. For example, sensed events (e.g., R-waves) are utilized to detect one or more cardiac episodes. In some variations, electrical circuitry 400 includes a processor is utilized to receive information regarding the sensed events and implements one or more algorithms for determining whether a particular one or more events have occurred. In addition, the analog voltage signals received from electrodes 304 and 306 may be passed to analog-to-digital (A/D) converters included in the electrical circuitry 400, and stored in a memory unit 406 included as part of electrical circuitry 400 for subsequent analysis with firmware executed by the processor(s) 404 included as part of electrical circuitry 400.

Electrical circuitry 400 may control insertable cardiac monitor 300 functions and process EGM signals received from electrodes 304 and 306 according to programmed signal analysis routines or algorithms. The insertable cardiac monitor 300 may include other optional sensors (not shown) for monitoring physiological signals, such as an activity sensor, pressure sensor, oxygen sensor, accelerometer, and/or other sensor used to monitor a patient. These may also be provided to electrical circuitry 400 for processing.

Electrical circuitry 400 may similarly control monitoring time intervals and sampling rates according to a particular clinical application. In addition, electrical circuitry may include state machines or other sequential logic circuitry to control device functions and need not be implemented exclusively as a microprocessor. For example, electrical circuitry 400 may include timers utilized to detect asystole events as described in more detail below.

Electrical circuitry 400 communicates with integrated antenna 322 (shown in FIG. 3) or other communication to transmit electrical signal data, e.g. EGM signal data, stored in memory or received from electrical circuitry 400 in real time. Antenna 322 may be configured to transmit and receive communication signals via inductive coupling, electromagnetic coupling, tissue conductance, Near Field Communication (NFC), Radio Frequency Identification (RFID), BLUETOOTH®, WiFi, or other proprietary or non-proprietary wireless telemetry communication schemes.

The electrical circuitry 400 may include a communication module including the integrated antenna 322, so as to enable the insertable cardiac monitor 300 to communicate with one or more external devices located external to the device 300. For example, as shown in FIG. 5, a cardiac monitoring system 500 may include an insertable cardiac monitor 10 (e.g., of which insertable cardiac monitor 300 is an example), which may include a communication module for communicating with a programmer 510. The programmer 510 may include a user interface that presents information to and receives input from a user. In some variations, the programmer 510 may include, for example, a suitable computing device such as a tablet, a smartphone, desktop computer, laptop computer, and/or the like. It should be noted that the user may also interact with programmer remotely via a networked computing device. As further shown in FIG. 5, in some variations, the insertable cardiac monitor 10 and/or the programmer 510 may be configured to transfer and/or receive information (e.g., cardiac data, such as EGM data and/or cardiac episode-related information derived from the EGM data) to and/or from a secondary memory storage device 520, such as over a wired or wireless network.

A user, such as a physician, technician, surgeon, electrophysiologist, other clinician, or patient, interacts with programmer to communicate with insertable cardiac monitor 300. For example, the user may interact with programmer to retrieve physiological or diagnostic information from the insertable cardiac monitor 300. A user may also interact with programmer to program the insertable cardiac monitor 300, e.g., select values for operational parameters of the insertable cardiac monitor 300. For example, the user may use programmer to retrieve information from the insertable cardiac monitor 300 regarding the rhythm of a patient heart, trends therein over time, or arrhythmic episodes. In some variations, alerts regarding device status (e.g., health state) and/or regarding type(s) of cardiac episode(s) detection may be provided to the patient or a clinician through the programmer 510, though they may be provided in any suitable manner (e.g., personal smartphone, other computing device, pushed through to an electronic medical record, etc.). The insertable cardiac monitor 300 and the programmer may communicate via wireless communication using any techniques known in the art.

In some variations, the insertable cardiac monitor 300 (which is an example of insertable cardiac monitor 10) can be placed subcutaneously in a patient near or over the patient's heart. For example, in some variations the insertable cardiac monitor 300 can be placed in a subcutaneous pocket located over an intercostal space (e.g., over the 4^{th} intercostal space), and positioned at a desirable angle and/or displacement relative to the patient's sternum (e.g., between about 0 and 45 degrees relative to the sternum, about 2 cm from the left edge of the sternum). Once inserted, the insertable cardiac monitor 300 may go through suitable setup and/or calibration processes.

Depiction of different features as modules is intended to highlight different functional aspects and does not necessarily imply that such modules must be realized by separate hardware or software components. Rather, functionality associated with one or more modules may be performed by separate hardware, firmware and/or software components, or integrated within common hardware, firmware and/or software components.

Furthermore, it should be understood that the systems and methods described herein in accordance with the present technology are not limited to the cardiac monitor 300 described herein with respect to FIGS. 3 and 4. Rather, the systems and methods described herein in accordance with the present technology may additionally or alternatively be used in conjunction with other cardiac monitor devices (e.g., other leadless cardiac monitoring devices, cardiac monitoring devices with leads, etc.).

### II. Methods for Dynamically Tuning Optical Sensing

In some instances, the implantable optical device can be an insertable cardiac monitor (e.g., device 200 or 300) that is implantable in a subcutaneous pocket. The insertable cardiac monitor may be configured such that the emitter and detector of an emitter-detector pair for generating a PPG measurement are oriented to face outward (e.g., facing away from muscle) such that the optical path of light from the emitter to the detector is through vascular tissue of interest. However, in some instances the implanted device may at least partially flip within the subcutaneous pocket (e.g., during a period after implantation and before encapsulation occurs, when the device may be less anchored in the tissue of the patient), which may lead to the emitter-detector pair not properly interfacing with the tissue of interest, and thus resulting in non-optimal PPG measurements. Such device flipping may, for example, occur as a result of natural migration, patient manipulation of the device (e.g., Twiddler's syndrome), etc.

Furthermore, implantable optical devices (e.g., device 200 or 300) are subject to optical noise conditions including, for example, ambient light shining into the patient body, device movement (e.g., as described above), patient movement, and/or variation of tissue properties in the patient (e.g., due to local anatomy, skin tone differences, etc.) resulting from patient-to-patient variations and/or changes in tissue over time.

As described herein, methods in accordance with the present technology enable the dynamic self-tuning of an optical sensing device, such as an implantable optical sensing device. For example, methods described herein enable the self-tuning of optical circuitry (e.g., associated with the emitter and/or detector of an emitter-detector pair for generating a PPG signal) to adapt to different levels of optical input due to device orientation and/or noise condition(s). Thus, methods described herein relate to self-detection of device orientation, and/or techniques for self-adjusting optical sensing in response to the device orientation and/or detected optical noise condition(s).

FIG. 1 is a flowchart schematic of an example method 100 for dynamic self-tuning of an implanted optical device. As shown in FIG. 1, a method 100 may include receiving at least one sensor signal from one or more sensors of an implanted optical device 110, detecting an orientation of the optical device based on the at least one sensor signal 120, and detecting an optical noise condition for the optical device 130. Although FIG. 1 illustrates both detecting an orientation of the optical device 120 and detecting an optical noise condition for the optical device 130, it should be understood that in some variations, the method 100 may include detecting an orientation of the optical device 120 without detecting an optical noise condition for the optical device 130, or may include detecting an optical noise condition for the optical device 130 without detecting an orientation of the optical device 120. The method 10 may further include modifying one or more characteristics of optical circuitry associated with an optical sensor arrangement in the optical device 140, based on the detected orientation of the device and/or the detected optical noise condition.

In some variations, the method 100 may be performed in a calibration or setup procedure, such as upon implantation or initialization of the optical sensing device. Additionally or alternatively, the method 100 may be performed intermittently or periodically at regular intervals, such as in a continual effort to make sure the optimal optical circuitry settings are being used to obtain PPG measurements. For example, the method may be performed approximately once per minute, once every 5 minutes, once every 10 minutes, once every 30 minutes, once every hour, or once every day, once every 5 cardiac cycles, once every 10 cardiac cycles, once every 30 cardiac cycles, once every 50 cardiac cycles, etc. Additionally or alternatively, aspects of the method 100 may additionally or alternatively be performed in response to a trigger event, such as a user command (e.g., through a programmer such as programmer 510), detection of a change in position of the optical sensing device relative to the patient (e.g., device orientation such as a device rotating or flipping within a subcutaneous pocket in the patient tissue), detection of patient movement (e.g., detected by an accelerometer in the optical sensing device or associated secondary device), and/or change in detected ambient light (e.g., detected by a light sensor located on the optical sensing device, detected by a secondary external wearable device with a light sensor in communication with the optical sensing system, etc.).

Receiving at least one sensor signal from one or more sensors of the implanted optical device 110 functions to gather sensor information from which a device status such as device orientation may be determined. In some variations, the one or more sensors in the optical device may include an optical sensor (e.g., in an optical sensor arrangement, such as optical sensor arrangement 210 or 410), an impedance sensor, and/or an accelerometer. In some variations, the method may further include receiving at least one sensor signal from a sensor in an external device. For example, information from an accelerometer can be received from a wearable device (e.g., smartwatch, pedometer, etc.) and may be analyzed with one or more suitable algorithms to determine patient posture, movement, activity levels, activity types, etc.

Detecting an orientation of the optical device 120 and detecting an optical noise condition 130 function to analyze the sensor data from the implanted optical device and/or associated external device(s) to assess whether there is a condition that may warrant modification of optical circuitry settings for improving the quality of a measurement PPG signal.

For example, in some variations, the optical sensor used to generate a PPG signal may provide sensor information indicative of device orientation. In some variations, the method may include detecting attenuation of the PPG signal over time, which may be caused by light from the emitter being transmitted through different types of tissue with varying signal attenuation properties. For example, the strength (e.g., amplitude) of a PPG signal may change depending on whether the emitter-detector pair of the optical device is facing subcutaneous fat, or facing muscle, since subcutaneous fat and muscle inherently have different properties. Accordingly, in some variations a sudden or gradual change in the overall strength of the PPG signal may be indicative of a change in device orientation.

As another example, an optical sensor (which may be the same as one used to generate a PPG signal, or a different optical sensor) may be used to measure ambient light levels throughout the day, and changes and/or general pattern in detected ambient light may be indicative of device movement within the patient. For example, the method may include evaluating detected ambient light over a 24-hour period. If the ambient light-detecting optical sensor is located on a surface of the optical device that is intended to be facing into the body, it may be expected that the optical sensor will generally consistently detect, on average, a low level of ambient light throughout the majority of the day. However, if the implanted optical device flips, then the optical sensor will likely begin detecting, on average, a higher level of ambient light throughout the majority of the day. Thus, in this example, an unexpected change from low ambient light to higher ambient light may be indicative of a flipped optical device. Similarly, if the ambient light-detecting optical sensor is located on a surface of the optical device that is intended to be facing outward from the body, it may be expected that the optical sensor will generally consistently detect, on average, a high level of ambient of light throughout the majority of the day. However, if the implanted optical device flips, then this optical sensor will likely begin detecting, on average, a lower level of ambient light throughout the majority of the day. Thus, in this example, an unexpected change from high ambient light to lower ambient light may be indicative of a flipped optical device.

As another example, ambient light levels over the course of a day may be evaluated to determine device orientation. If the ambient light-detecting optical sensor is located on a surface of the optical device that is intended to be facing into the body, then it may be expected that over the course of a 24-hour period, the optical sensor will generally consistently detect, on average, a low level of ambient light throughout the 24-hour period. However, if the optical sensor detects greater levels of ambient light during daytime than nighttime over the 24-hour, this may be indicative of a flipped optical device. Although this example is described with respect to a 24-hour period, it should be understood that the ambient light levels may be evaluated over any suitable period of time (e.g., sunrise to sunset, any period of time when the patient is determined to be awake or active, such as through accelerometer data from the optical device and/or external device).

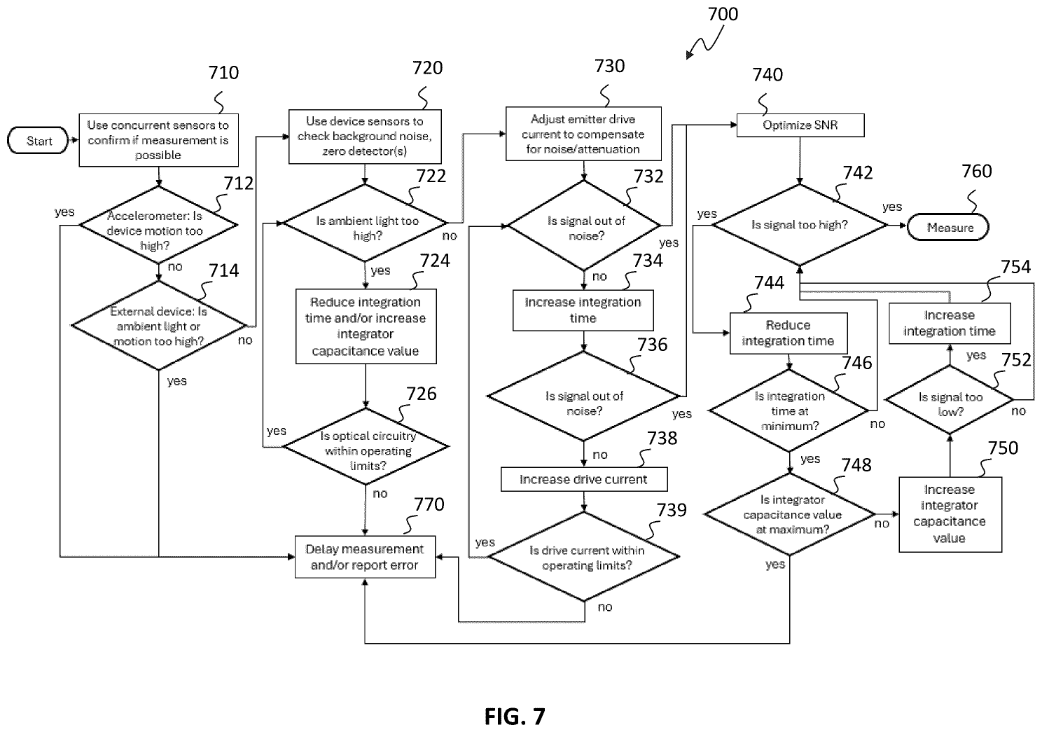

Furthermore, the optical sensor signal may be used to evaluate for noise conditions. For example, the integrated optical sensor signal can be compared to a predetermined threshold to check that the integrated optical signal is stronger than an assumed background noise level associated with ambient light, tissue-based signal attenuation, etc. (e.g., has a sufficient signal to noise ratio). Further details regarding this evaluation are described below with respect to FIG. 7.

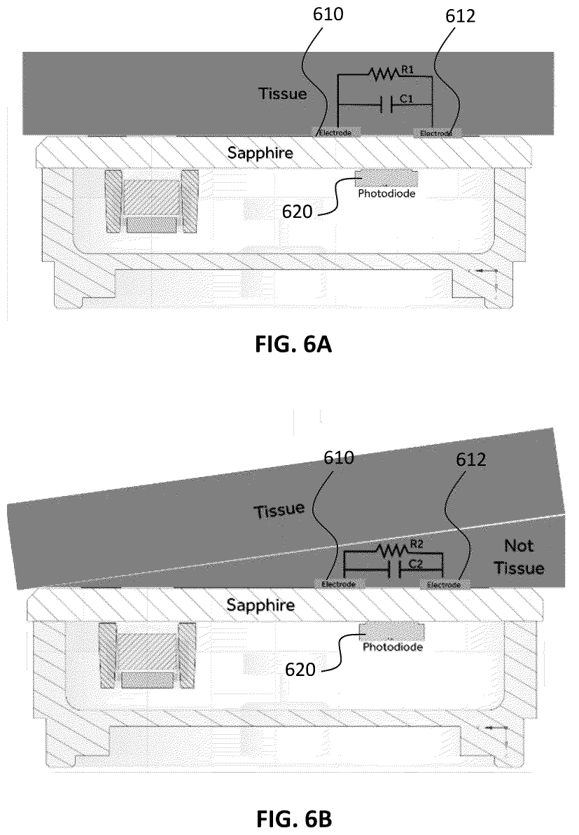

Additionally or alternatively, in some variations, the optical device may include an impedance sensor comprising at least two electrodes, which may be located proximate to the optical transmission path(s) extending between emitter-detector pairs on the optical device and may be isolated from electrodes used to measure cardiac electrical signals. For example, as shown in FIGS. 6A and 6B, an optical device may include a first electrode 610 and a second electrode 612 that are arranged over an outer surface of the optical device, in a region overlying a detector 620 that can be used to generate PPG measurements. In FIG. 6A, the optical device is oriented relative to tissue of interest (“tissue”) with the detector 620 configured to interface with (e.g., receive light transmitted through) the tissue of interest. As shown in FIG. 6A, a first impedance measurement path between the first and second electrodes 610, 612 is also located in the tissue of interest, such that the impedance measurement path is characterized by a resistance R1 and a capacitance C1 associated with the tissue of interest. In contrast, FIG. 6B depicts the optical device as oriented with the detector 620 configured to interface with anatomy not including the tissue of interest (“not tissue”). As shown in FIG. 6B, a second impedance measurement path between the first and second electrodes 610, 612 is not in the tissue of interest, such that the impedance measurement path is characterized by a resistance R2 and a capacitance C2 associated with other anatomy not including the tissue of interest. Accordingly, the first and second impedance measurement paths will produce different impedance measurements that are each indicative of the different tissue interfaces between the optical device and adjacent to tissue. In operation, the first and second electrodes 610, 612 may be used to obtain an impedance measurement that can be compared to a known threshold or range of known impedance of the tissue of interest, and thus impedance measurements may be used to help determine state of a tissue interface with the optical device and/or an orientation of the optical device in tissue (e.g., a device flip).

In some variations, accelerometer data may be used to detect a direction of gravity, which may be indicative of patient posture and/or orientation which may be correlated to a device orientation. For example, if a patient is assumed to be upright for a majority of the day, but the accelerometer data suggests that the patient is prone for a majority of the day, then this may be an indication of a change in device orientation.

Additionally or alternatively, in some variations, the method may include receiving a user input relating to a device orientation check. For example, in some variations, the optical device may include a light emitter configured to emit light outwards from the patient so that the light is visible through the skin surface. In some variations, as part of a device orientation check, the user may enter a confirmation that they can see the light through the skin surface as expected (e.g., in response to a prompt). The device orientation check may be performed, for example, upon implantation of the optical device, periodically or intermittently after implantation, in response to one or more trigger events such as a suspected device flip that is detected by one or more techniques described herein, or in response to a check triggered by a user (e.g., patient, clinician, etc.), or any combination thereof. In some variations, such a light emitter may be activated only as part of a device check. However, in some variations the light emitter may consistently or continuously be activated, such that the absence of visible light through the skin is immediately recognizable as indicative of a likely flipped device.

Modifying one or more characteristics of optical circuitry 140 functions to adjust optical sensor settings to optimize the detector signal for the given detected device orientation and/or optical noise conditions. For example, in some variations, the method may include adjusting the drive current for an active emitter in an emitter-detector pair intended to be used for PPG measurements. The drive current may be increased to boost the amplitude of the emitted light and resulting detector signal, and may be reduced to conserve power and extend battery life. As another example, in some variations, the method may additionally or alternatively include adjusting integration time in an integrator amplifier circuit associated with the active detector in an emitter-detector pair intended to be used for PPG measurements. The integration time may be increased to increase the signal gain and boost the amplitude of the integrated optical sensor signal, and may be reduced to keep the integrated optical sensor signal within a manageable range. Furthermore, in some variations, the method may additionally or alternatively include adjusting a capacitor value of an integrating capacitor in an integrator amplifier circuit associated with the active detector, thereby adjusting integration sensitivity. The integrator capacitance value may be reduced to increase the signal gain and boost the amplitude of the integrated optical sensor signal, and may be reduced to keep the integrated optical sensor signal within a manageable range.

For example, the optical sensor signal may be compared to a lower threshold and an upper threshold to help ensure that the optical sensor signal is within a desired range. If the optical sensor signal is either below the lower threshold or above the upper threshold, the drive current, the integration time and/or the integrator capacitance value may be adjusted (e.g., in an iterative manner) until the integrated optical sensor signal is within the desired range.

In some variations, the method may additionally or alternatively include selecting a different emitter-detector pair for use in generating PPG measurements, such as in response to a detected change in device orientation. For example, as described above, in some variations the optical device has a first emitter-detector pair on a first optical device surface (e.g., configured to be outward-facing) and a second emitter-detector pair on a second optical device surface (e.g., configured to be inward-facing) opposite the first optical device surface. In these variations, the method may include transitioning from use of the first emitter-detector pair to the use of the second emitter-detector pair, in response to detection of a device flip. Other emitter-detector pairs may also be selected in response to other kinds of changes in device orientation. Additionally or alternatively, in some variations the method may include ceasing generation of PPG measurements in response to a change in device orientation (e.g., device flip), such as if it is determined that an otherwise-active emitter-detector pair is facing inwards toward muscle tissue and is not suitable to detect pulsatile activity for PPG measurements, and no secondary emitter-detector pair is available or desired to be selected.

Additionally or alternatively, in some variations the method may include providing an alert to a user (e.g., patient, clinician, etc.) indicating when no suitable optical circuitry settings have been found through the dynamic self-tuning process, such as when optical noise conditions are too excessive. The alert may be provided, for example, to a suitable programmer or other computing device (e.g., programmer 510), and may indicate any suitable level of specificity. For example, in some variations the alert may be a general indication of a failure to obtain a PPG measurement, while in some variations the alert may include a predicted specific reason for the failure to obtain a PPG measurement.

Furthermore, in some variations the method may include providing an alert to a user (e.g., patient, clinician, etc.) indicating a flipped optical device or other change in device orientation, with or without performing dynamic self-tuning described herein. Such an alert may, for example, be useful to prompt a clinician to perform corrective actions such as repositioning the optical device (e.g., a corrective device flip within the subcutaneous pocket).

### II. Example

FIG. 7 illustrates an example method 700 of self-tuning of an optical device. Although the processes are depicted in a certain order, in other variations they may be performed in any suitable order.

As shown in FIG. 7, however, a threshold evaluation in method 700 includes the use of sensors concurrent to optical sensors that are configured to confirm whether a PPG measurement is possible (block 710) or other appropriate to provide a minimum quality of PPG signal. For example, an accelerometer in the optical device may provide sensor data indicative of device motion, and the method 700 may include determining whether the amount of device motion is too high (e.g. above a predetermined threshold) (block 712). If so, this may be indicative of the patient motion being too high to obtain a quality PPG signal, in which case PPG measurement may be delayed and/or an error may be reported to the patient (or clinician, etc.) such as through a computing device (e.g., programmer 510) drawing attention to the determination that conditions are not suitable for PPG measurement (block 770). Even if the accelerometer onboard the optical device does not determine that the device motion is not too high, the method may further include using sensor data from an external device (e.g., external accelerometer) to similarly check whether device (and patient) motion is too high (block 714). Additionally or alternatively, an external device with a light sensor may separately provide sensor data regarding ambient light levels, and the method 700 may further include determining from such external light sensor data whether the ambient light is too high (block 714), such as by comparing the external light sensor data with a predetermined threshold. If the external light sensor data indicates that device motion is too high, PPG measurement may similarly be delayed and/or an error may be reported to alert that conditions are not suitable for PPG measurement (block 770).

If the threshold evaluation described above indicates PPG measurement is possible, then device sensors may be used to check background noise and/or zero the detector used for PPG measurement (block 720). To check for background signal, the method may include turning off any emitter(s) in the optical device and operating the detector to solely detect light coming from external or ambient sources. The signal from the active detector under such conditions may be used to determine whether the background noise (e.g., ambient light) is too high for the given optical circuitry settings (block 722). If so, then various optical circuitry settings may be changed until the background noise results in effectively a “zero” setting for the detector. For example, the method may include changing (e.g., reducing or increasing) integration time and/or changing (e.g., increasing or reducing) integrator capacitance value (block 724), thereby reducing the detector signal output. In an example implementation, integration time may be reduced and the integrator capacitance value may be increased across one or more iterations until background noise is read at effectively zero by the detector, as long as these parameters are within operating limits of the optical circuitry (block 726). In some variations, between each iteration the integration time may be halved and the integrator capacitance value may be doubled, though the integration time and integrator capacitance value may be adjusted but any suitable increment between each iteration. If the optical circuitry parameters cannot be adjusted enough to zero the detector without being outside operating limits, then the PPG measurement may be delayed and/or an error may be reported (block 770) as described above.

Once the detector is zeroed against background noise, the method may further include adjusting drive current to the emitter to be used to generate a PPG signal (block 730), which adjusts (e.g., increases) the intensity of the emitter to attempt to compensate for optical noise and/or other attenuation of the detector signal. Generally, the drive current and/or integration time may be adjusted until the integrated optical sensor signal is at least above a certain threshold to help ensure that the integrated optical sensor signal is above the noise level. To optimize power consumption and increase battery life, it may be advantageous to drive the emitter at an intensity as low as possible; accordingly, the method may include first checking whether the detector signal is sufficiently stronger than background noise while operating the emitter at a minimum operating level (e.g., with a minimum drive current, such as 1 mA). For example, drive current may be considered sufficiently high if the integrated optical sensor signal is at least a predetermined minimum threshold (e.g., at least 0.25V). If the signal is not at a sufficiently high level to be above or out of background noise (block 732), then the method may further include increasing the integration time to a maximum value without yet further adjusting drive current (block 734) and checking against whether the signal is sufficiently stronger than background noise (block 736). If increasing the integration time to a maximum value is not enough to make the signal sufficiently stronger than background noise, then drive current may be increased (block 738) as long as the drive current is still within operating limits (block 739). In this manner, drive current may be iteratively increased and evaluated until a certain level of drive current is found for resulting in a detector signal that is sufficiently above or out of background noise. In some variations, the incremental step for evaluating drive currents may involve doubling the drive current value at each iteration though any suitable incremental step may be used. If no sufficient drive current value is found, then the PPG measurement may be delayed and/or an error may be reported (block 770) as described above.

When a suitable drive current has been found, then signal to noise ratio (SNR) may be optimized (block 740) by iteratively adjusting integration time and integrator capacitance value until the integrated optical sensor signal under the circumstances is within a certain range. The minimum end of the range may be related to the predetermined minimum threshold discussed above with respect to block 732 (e.g. 0.25V), while the maximum end of the range may be a predetermined maximum threshold (e.g., 0.5V), though any suitable values may be used. As shown in FIG. 7, if the integrated optical sensor signal is too high (block 742) and above the predetermined maximum threshold, then the integration time may be reduced (block 744) and/or the integrator capacitance value may be increased (block 750) in an iterative manner until the integrated optical sensor signal is within the desired range. With each iteration, the method may include checking that the integration time is not reduced below a minimum threshold (block 746) and checking that the integrator capacitance value is not increased above a maximum operating threshold (block 748). The method may further include checking that these adjustments to make integration time and/or integrator capacitance value fall below the predetermined maximum threshold do not inadvertently push the integrated optical sensor signal below the predetermined minimum threshold (block 752); if so then the integration time may be increased again (block 754). Once a suitable integration time and integrator capacitance value are found, then the optical circuitry settings (including the drive current, the integration time, and the integrator capacitance determined as described above) may be used to obtain PPG measurements (block 760). However, if no suitable integration time and/or integrator capacitance value are found, then the PPG measurement may be delayed and/or an error may be reported (block 770) as described above.

### Conclusion

Although many of the variations are described above with respect to systems, devices, and methods for operating an implantable cardiac monitoring device, the technology is applicable to other applications and/or other approaches, such as other implantable optical sensing device for other purposes. Moreover, other variations in addition to those described herein are within the scope of the technology. Additionally, several other variations of the technology can have different configurations, components, or procedures than those described herein. A person of ordinary skill in the art, therefore, will accordingly understand that the technology can have other variations with additional elements, or the technology can have other variations without several of the features shown and described above with reference to FIG. 1-7.

The descriptions of variations of the technology are not intended to be exhaustive or to limit the technology to the precise form disclosed above. Where the context permits, singular or plural terms may also include the plural or singular term, respectively. Although specific variations of, and examples for, the technology are described above for illustrative purposes, various equivalent modifications are possible within the scope of the technology, as those skilled in the relevant art will recognize. For example, while steps are presented in a given order, alternative variations may perform steps in a different order. The various variations described herein may also be combined to provide further variations.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific variations have been described herein for purposes of illustration, but that various modifications may be made without deviating from the technology. Further, while advantages associated with certain variations of the technology have been described in the context of those variations, other variations may also exhibit such advantages, and not all variations need necessarily exhibit such advantages to fall within the scope of the technology. Accordingly, the disclosure and associated technology can encompass other variations not expressly shown or described herein.

1. A method of operating an optical device implanted in a patient, the method comprising:
receiving at least one sensor signal from one or more sensors of the implanted optical device;
detecting an orientation of the optical device based on the least one sensor signal; and
based on the detected orientation of the optical device, modifying one or more characteristics of optical circuitry associated with an optical sensor arrangement in the optical device, wherein the optical sensor arrangement is configured to generate a photoplethysmography (PPG) signal for the patient.

2. The method of claim 1, wherein the at least one sensor signal comprises an optical sensor signal from the optical sensor arrangement of the optical device.

3. The method of claim 2, wherein the optical sensor signal comprises a photoplethysmography (PPG) signal, and wherein detecting an orientation of the optical device comprises detecting an orientation of the optical device based on the PPG signal.

4. The method of claim 2, wherein detecting an orientation of the optical device comprises evaluating ambient light level outside of the patient based on the at least one optical sensor signal.

5. The method of claim 1, wherein the one or more sensors comprises an impedance sensor, wherein the at least one sensor signal comprises an impedance sensor signal from the impedance sensor, and wherein detecting an orientation of the optical device comprises evaluating a type of tissue contacting the optical device based on the impedance sensor signal.

6. The method of claim 1, wherein receiving at least one sensor signal comprises receiving an accelerometer signal, and wherein the method further comprises evaluating optical sensing conditions based on the accelerometer signal.

7. The method of claim 1, wherein modifying one or more characteristics of optical circuitry comprises modifying a drive current associated with an emitter of the optical sensor arrangement.

8. The method of claim 1, wherein modifying one or more characteristics of optical circuitry comprises modifying an integration capacitor value in an integrator amplifier of the optical circuitry.

9. The method of claim 1, wherein modifying one or more characteristics of optical circuitry comprises modifying an integration time value in an integrator amplifier of the optical circuitry.

10. The method of claim 1, further comprising generating a measurement PPG signal for the patient using the modified optical circuitry.

11. An implantable cardiac monitoring device, comprising:
one or more sensors;
a processor;
a memory operably coupled to the processor and storing instructions that, when executed by the processor, cause the cardiac monitoring device to perform operations comprising:
receiving at least one sensor signal from the one or more sensors when the cardiac monitoring device is implanted in a patient;
detecting an orientation of the cardiac monitoring device based on the least one sensor signal; and
based on the detected orientation of the cardiac monitoring device, modifying one or more characteristics of optical circuitry associated with an optical sensor arrangement in the cardiac monitoring device, wherein the optical sensor arrangement is configured to generate a photoplethysmography (PPG) signal for the patient.

12. The device of claim 11, wherein the at least one sensor signal comprises an optical sensor signal from the optical sensor arrangement of the cardiac monitoring device.

13. The system of claim 11, wherein the optical sensor signal comprises a photoplethysmography (PPG) signal, and wherein detecting an orientation of the cardiac monitoring device comprises detecting an orientation of the cardiac monitoring device based on the PPG signal.

14. The system of claim 12, wherein detecting an orientation of the cardiac monitoring device comprises evaluating ambient light level outside of the patient based on the at least one optical sensor signal.

15. The system of claim 11, wherein the one or more sensors comprises an impedance sensor, wherein the at least one sensor signal comprises an impedance sensor signal from the impedance sensor, and wherein detecting an orientation of the cardiac monitoring device comprises evaluating a type of tissue contacting the cardiac monitoring device based on the impedance sensor signal.

16. The system of claim 11, wherein receiving at least one sensor signal comprises receiving an accelerometer signal, and wherein the method further comprises evaluating optical sensing conditions based on the accelerometer signal.

17. The system of claim 11, wherein modifying one or more characteristics of optical circuitry comprises modifying a drive current associated with an emitter of the optical sensor arrangement.

18. The system of claim 11, wherein modifying one or more characteristics of optical circuitry comprises modifying an integration capacitor value in an integrator amplifier of the optical circuitry.

19. The system of claim 11, wherein modifying one or more characteristics of optical circuitry comprises modifying an integration time value in an integrator amplifier of the optical circuitry.

20. The system of claim 11, further comprising generating a measurement PPG signal for the patient using the modified optical circuitry.
